(19) United States
(12) Patent Application Publication
(10) Pub. No.: US 2026/0224851 A1
(43) Pub. Date: Aug. 6, 2026

(54) TOOL BASED FLEXIBLE ELONGATE DEVICE CONTROL

(71) Applicant: INTUITIVE SURGICAL OPERATIONS, INC., Sunnyvale, CA (US)

(72) Inventors: Shibing Liu, San Jose, CA (US); Samuel B. Schorr, San Jose, CA (US)

(21) Appl. No.: 19/159,156

(22) PCT Filed: Feb. 21, 2024
(86) PCT No.: PCT/US24/16622

§ 371 Date: Aug. 22, 2025

Related U.S. Application Data
(60) Provisional application No. 63/486,489, filed on Feb. 23, 2023.

(51) Int. Cl. A61M 25/01; A61M 25/06

(52) U.S. Cl. CPC A61M 25/0147; A61M 2025/0166; A61M 2025/0681

(57) ABSTRACT

Medical systems and methods include a flexible elongate device having an articulable body portion and an actuator configured to control articulation of the articulable body portion of the flexible elongate device. A control system determines an expected position of the articulable body portion, determines tool data associated with a tool insertable within a lumen of the flexible elongate device, determines adjustment data based on a model of the flexible elongate device with the tool data to cause an actual position of the articulable body portion to correspond with the expected position when the tool is inserted within the lumen, and operates the actuator to control articulation the articulable body portion according to the adjustment data.

### FIELD

Disclosed embodiments relate to assemblies for a flexible elongate device.

### BACKGROUND

Minimally invasive medical techniques are intended to reduce the amount of tissue that is damaged during medical procedures, thereby reducing patient recovery time, discomfort, and harmful side effects. Such minimally invasive techniques may be performed through natural orifices in a patient anatomy or through one or more surgical incisions. Through these natural orifices or incisions, physicians may insert minimally invasive medical instruments (including surgical, diagnostic, therapeutic, and/or biopsy instruments) to reach a target tissue location. One such minimally invasive technique is to use a flexible and/or steerable elongate device, such as a flexible catheter, that can be inserted into anatomic passageways and navigated toward a region of interest within the patient anatomy.

### SUMMARY

The following presents a simplified summary of various examples described herein and is not intended to identify key or critical elements or to delineate the scope of the claims.

According to some embodiments, a medical system includes a flexible elongate device having a lumen extending therethrough for receiving one or more tools and having an articulable body portion, an actuator configured to control articulation of the articulable body portion of the flexible elongate device, and a control system. The control system is configured to: determine an expected position of the articulable body portion of the flexible elongate device, determine tool data associated with a tool insertable within the lumen of the flexible elongate device, determine adjustment data based on a model of the flexible elongate device with the tool data to cause an actual position of the articulable body portion of the flexible elongate device to correspond with the expected position when the tool is inserted within the lumen, and operate the actuator to control articulation of the articulable body portion of the flexible elongate device according to the adjustment data.

According to some embodiments, a method for controlling a flexible elongate device includes determining an expected position for an articulable body portion of a flexible elongate device, the flexible elongate device having a lumen extending therethrough for receiving one or more tools, determining tool data associated with a tool insertable within the lumen of the flexible elongate device, determining adjustment data based on a model of the flexible elongate device with the tool data to cause an actual position of the articulable body portion of the flexible elongate device to correspond with the expected position when the tool is inserted within the lumen, and operating an actuator to control articulation of the articulable body portion of the flexible elongate device according to the adjustment data.

It is to be understood that both the foregoing general description and the following detailed description are illustrative and explanatory in nature and are intended to provide an understanding of the present disclosure without limiting the scope of the present disclosure. In that regard, additional aspects, features, and advantages of the present disclosure will be apparent to one skilled in the art from the following detailed description.

### BRIEF DESCRIPTIONS OF THE DRAWINGS

FIG. 1 is a simplified diagram of a medical system according to some embodiments.

FIG. 2A is a simplified diagram of a medical instrument system according to some embodiments.

FIG. 2B is a simplified diagram of a medical instrument including a medical tool within an elongate device according to some embodiments.

FIGS. 3A and 3B are simplified diagrams of side views of a patient coordinate space including a medical instrument mounted on an insertion assembly according to some embodiments.

FIG. 4 is a simplified diagram of a medical system according to some embodiments.

FIG. 5 is a flowchart for operating a medical system according to some embodiments.

FIG. 6 is a flowchart for operating a medical system according to some embodiments.

FIG. 7 is a flowchart for a method of operating a medical system according to some embodiments.

Embodiments of the present disclosure and their advantages are best understood by referring to the detailed description that follows. It should be appreciated that like reference numerals are used to identify like elements illustrated in one or more of the figures, wherein showings therein are for purposes of illustrating embodiments of the present disclosure and not for purposes of limiting the same.

### DETAILED DESCRIPTION

In the following description, specific details are set forth describing some embodiments consistent with the present disclosure. Numerous specific details are set forth in order to provide a thorough understanding of the embodiments. It will be apparent, however, to one skilled in the art that some embodiments may be practiced without some or all of these specific details. The specific embodiments disclosed herein are meant to be illustrative but not limiting. One skilled in the art may realize other elements that, although not specifically described here, are within the scope and the spirit of this disclosure. In addition, to avoid unnecessary repetition, one or more features shown and described in association with one embodiment may be incorporated into other embodiments unless specifically described otherwise or if the one or more features would make an embodiment non-functional. In some instances, well known methods, procedures, components, and circuits have not been described in detail so as not to unnecessarily obscure aspects of the embodiments.

This disclosure describes various instruments and portions of instruments in terms of their state in three-dimensional space. As used herein, the term “position” refers to the location of an object or a portion of an object in a three-dimensional space (e.g., three degrees of translational freedom along Cartesian x-, y-, and z-coordinates). As used herein, the term “orientation” refers to the rotational placement of an object or a portion of an object (e.g., one or more degrees of rotational freedom such as, roll, pitch, and yaw). As used herein, the term “pose” refers to the position of an object or a portion of an object in at least one degree of translational freedom and to the orientation of that object or portion of the object in at least one degree of rotational freedom (e.g., up to six total degrees of freedom). As used herein, the term “shape” refers to a set of poses, positions, and/or orientations measured along an object. As used herein, the term “distal” refers to a position that is closer to a procedural site and the term “proximal” refers to a position that is further from the procedural site. Accordingly, the distal portion or distal end of an instrument is closer to a procedural site than a proximal portion or proximal end of the instrument when the instrument is being used as designed to perform a procedure.

The disclosure relates to systems and methods to controlling the articulation of a flexible elongate device based on the properties of the tool inserted within the flexible elongate device. The insertion of different types of tools, having different physical properties, may result in changes to the articulation control of the flexible elongate device. For example, insertion of the tool may change the combined stiffness (e.g., as well as other physical properties that impact articulation control) of the flexible elongate device and tool. In another example, when a tool is inserted within the flexible elongate device, a position of the (e.g., distal) articulable body portion of the flexible elongate device may change in response to the tool insertion. To return the flexible elongate device back to a desired position prior to tool insertion, the articulation control accounts for how insertion of the tool impacts the articulation control.

In some embodiments, a shape sensor or other type of sensor may be used to directly measure the shape of the flexible elongate device when the tool is inserted, thereby providing a closed loop feedback articulation control. However, not all flexible elongate devices include sensors with sufficient capability for direct shape measurements, such sensors may degrade over time and become unreliable, or redundant sensing may be desirable to improve accuracy. As such, in some embodiments, an actuator encoder and model may be used for the articulation control. Here, the actuator encoder measures the activity of the actuators that are used to articulate the flexible elongate device and the model correlates the actuator activity with the shape of the flexible elongate device. The effectiveness of this articulation control depends on the quality and characteristics of the model. In some examples, the model used for articulation control may be updated based on the properties of different types of tools that may be inserted into the flexible elongate device.

Models for the systems and methods herein may include a stiffness of the tool that is used to correct the position of the articulable body portion of the flexible elongate device, such as a position of a tip of the flexible elongate device. For example, the model may be adjusted account for the increased stiffness in the flexible elongate device resulting from receiving the tool therethrough.

The systems and methods herein utilize properties, e.g., stiffness, inertia, friction, etc., associated with the tool to control the articulation of the flexible elongate device. For example, a medical system may include a flexible elongate device, an actuator, and a control system. The flexible elongate device includes an articulable body portion and a lumen extending through the flexible elongate device for receiving one or more tools. The actuator is configured to control articulation of the articulable body portion. The control system determines an expected position of the articulable body portion of the flexible elongate device; updates a model of the flexible elongate device based on the tool data; determines adjustment data for operating the actuator to cause an actual position of the articulable body portion to correspond with the expected position based on the model; and operates the actuator to control the articulable body portion according to the adjustment data.

The systems and methods disclosed herein improve positional accuracy and reduce tip position deviation for flexible elongate devices, such as catheters, that may result when a tool or other object is inserted into the flexible devices. Reducing tip position deviation improves user experience and increases system reliability. For example, the systems and methods may allow a user to continue a procedure that would previously have been aborted due to poor tip control. If a shape sensor provides an error or otherwise degrades to an undesirable level, the control may be switched to utilizing encoder feedback to allow the user to continue a procedure.

In some embodiments, a shape sensor or other type of sensor capable of directly measuring the shape of the flexible elongate device may be used to update the model. The update may include changes to the tool properties of a tool that is inserted into the flexible elongate device. The tool properties may be stored in association with the tool type for subsequent use with the model. While the sensor is available, the resulting measurements may also be used to provide a closed loop feedback articulation control. When the sensor becomes unavailable, the (e.g., most recent) updated model may be used along with data from the actuator encoder to provide the articulation control. When the tool within the flexible elongate device is replaced with another tool, previously stored tool properties of the new tool may be used to update the model.

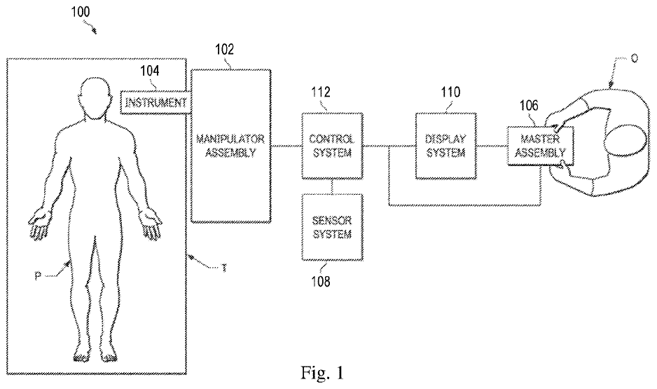

FIG. 1 is a simplified diagram of a medical system 100 according to some embodiments. The medical system 100 may be suitable for use in, for example, surgical, diagnostic (e.g., biopsy), or therapeutic (e.g., ablation, electroporation, etc.) procedures. While some embodiments are provided herein with respect to such procedures, any reference to medical or surgical instruments and medical or surgical methods is non-limiting. The systems, instruments, and methods described herein may be used for animals, human cadavers, animal cadavers, portions of human or animal anatomy, non-surgical diagnosis, as well as for industrial systems, general or special purpose robotic systems, general or special purpose teleoperational systems, or robotic medical systems.

As shown in FIG. 1, medical system 100 may include a manipulator assembly 102 that controls the operation of a medical instrument 104 in performing various procedures on a patient P. Medical instrument 104 may extend into an internal site within the body of patient P via an opening in the body of patient P. The manipulator assembly 102 may be teleoperated, non-teleoperated, or a hybrid teleoperated and non-teleoperated assembly with one or more degrees of freedom of motion that may be motorized and/or one or more degrees of freedom of motion that may be non-motorized (e.g., manually operated). The manipulator assembly 102 may be mounted to and/or positioned near a patient table T. A master assembly 106 allows an operator O (e.g., a surgeon, a clinician, a physician, or other user) to control the manipulator assembly 102. In some examples, the master assembly 106 allows the operator O to view the procedural site or other graphical or informational displays. In some examples, the manipulator assembly 102 may be excluded from the medical system 100 and the instrument 104 may be controlled directly by the operator O. In some examples, the manipulator assembly 102 may be manually controlled by the operator O. Direct operator control may include various handles and operator interfaces for hand-held operation of the instrument 104.

The master assembly 106 may be located at a surgeon's console which is in proximity to (e.g., in the same room as) a patient table T on which patient P is located, such as at the side of the patient table T. In some examples, the master assembly 106 is remote from the patient table T, such as in in a different room or a different building from the patient table T. The master assembly 106 may include one or more control devices for controlling the manipulator assembly 102. The control devices may include any number of a variety of input devices, such as joysticks, trackballs, scroll wheels, directional pads, buttons, data gloves, trigger-guns, hand-operated controllers, voice recognition devices, motion or presence sensors, and/or the like.

The manipulator assembly 102 supports the medical instrument 104 and may include a kinematic structure of links that provide a set-up structure. The links may include one or more non-servo controlled links (e.g., one or more links that may be manually positioned and locked in place) and/or one or more servo controlled links (e.g., one or more links that may be controlled in response to commands, such as from a control system 112). The manipulator assembly 102 may include a plurality of actuators (e.g., motors) that drive inputs on the medical instrument 104 in response to commands, such as from the control system 112. The actuators may include drive systems that move the medical instrument 104 in various ways when coupled to the medical instrument 104. For example, one or more actuators may advance medical instrument 104 into a naturally or surgically created anatomic orifice. Actuators may control articulation of the medical instrument 104, such as by moving the distal end (or any other portion) of medical instrument 104 in multiple degrees of freedom. These degrees of freedom may include three degrees of linear motion (e.g., linear motion along the X, Y, Z Cartesian axes) and in three degrees of rotational motion (e.g., rotation about the X, Y, Z Cartesian axes). One or more actuators may control rotation of the medical instrument about a longitudinal axis. Actuators can also be used to move an articulable end effector of medical instrument 104, such as for grasping tissue in the jaws of a biopsy device and/or the like, or may be used to move or otherwise control tools (e.g., imaging tools, ablation tools, biopsy tools, electroporation tools, etc.) that are inserted within the medical instrument 104.

The medical system 100 may include a sensor system 108 with one or more sub-systems for receiving information about the manipulator assembly 102 and/or the medical instrument 104. Such sub-systems may include a position sensor system (e.g., that uses electromagnetic (EM) sensors or other types of sensors that detect position or location); a shape sensor system for determining the position, orientation, speed, velocity, pose, and/or shape of a distal end and/or of one or more segments along a flexible body of the medical instrument 104; a visualization system (e.g., using a color imaging device, an infrared imaging device, an ultrasound imaging device, an x-ray imaging device, a fluoroscopic imaging device, a computed tomography (CT) imaging device, a magnetic resonance imaging (MRI) imaging device, or some other type of imaging device) for capturing images, such as from the distal end of medical instrument 104 or from some other location; and/or actuator position sensors such as resolvers, encoders, potentiometers, and the like that describe the rotation and/or orientation of the actuators controlling the medical instrument 104.

The medical system 100 may include a display system 110 for displaying an image or representation of the procedural site and the medical instrument 104. Display system 110 and master assembly 106 may be oriented so physician O can control medical instrument 104 and master assembly 106 with the perception of telepresence.

In some embodiments, the medical instrument 104 may include a visualization system, which may include an image capture assembly that records a concurrent or real-time image of a procedural site and provides the image to the operator O through one or more displays of display system 110. The image capture assembly may include various types of imaging devices. The concurrent image may be, for example, a two-dimensional image or a three-dimensional image captured by an endoscope positioned within the anatomical procedural site. In some examples, the visualization system may include endoscopic components that may be integrally or removably coupled to medical instrument 104. Additionally or alternatively, a separate endoscope, attached to a separate manipulator assembly, may be used with medical instrument 104 to image the procedural site. The visualization system may be implemented as hardware, firmware, software or a combination thereof which interact with or are otherwise executed by one or more computer processors, such as of the control system 112.

Display system 110 may also display an image of the procedural site and medical instruments, which may be captured by the visualization system. In some examples, the medical system 100 provides a perception of telepresence to the operator O. For example, images captured by an imaging device at a distal portion of the medical instrument 104 may be presented by the display system 110 to provide the perception of being at the distal portion of the medical instrument 104 to the operator O. The input to the master assembly 106 provided by the operator O may move the distal portion of the medical instrument 104 in a manner that corresponds with the nature of the input (e.g., distal tip turns right when a trackball is rolled to the right) and results in corresponding change to the perspective of the images captured by the imaging device at the distal portion of the medical instrument 104. As such, the perception of telepresence for the operator O is maintained as the medical instrument 104 is moved using the master assembly 106. The operator O can manipulate the medical instrument 104 and hand controls of the master assembly 106 as if viewing the workspace in substantially true presence, simulating the experience of an operator that is physically manipulating the medical instrument 104 from within the patient anatomy.

In some examples, the display system 110 may present virtual images of a procedural site that are created using image data recorded pre-operatively (e.g., prior to the procedure performed by the medical instrument system 200) or intra-operatively (e.g., concurrent with the procedure performed by the medical instrument system 200), such as image data created using computed tomography (CT), magnetic resonance imaging (MRI), positron emission tomography (PET), fluoroscopy, thermography, ultrasound, optical coherence tomography (OCT), thermal imaging, impedance imaging, laser imaging, nanotube X-ray imaging, and/or the like. The virtual images may include two-dimensional, three-dimensional, or higher-dimensional (e.g., including, for example, time based or velocity-based information) images. In some examples, one or more models are created from pre-operative or intra-operative image data sets and the virtual images are generated using the one or more models.

In some examples, for purposes of imaged guided medical procedures, display system 110 may display a virtual image that is generated based on tracking the location of medical instrument 104. For example, the tracked location of the medical instrument 104 may be registered (e.g., dynamically referenced) with the model generated using the pre-operative or intra-operative images, with different portions of the model correspond with different locations of the patient anatomy. As the medical instrument 104 moves through the patient anatomy, the registration is used to determine portions of the model corresponding with the location and/or perspective of the medical instrument 104 and virtual images are generated using the determined portions of the model. This may be done to present the operator O with virtual images of the internal procedural site from viewpoints of medical instrument 104 that correspond with the tracked locations of the medical instrument 104.

The medical system 100 may also include the control system 112, which may include processing circuitry that implements the some or all of the methods or functionality discussed herein. The control system 112 may include at least one memory and at least one processor for controlling the operations of the manipulator assembly 102, the medical instrument 104, the master assembly 106, the sensor system 108, and/or the display system 110. Control system 112 may include instructions (e.g., a non-transitory machine-readable medium storing the instructions) that when executed by the at least one processor, configures the one or more processors to implement some or all of the methods or functionality discussed herein. While the control system 112 is shown as a single block in FIG. 1, the control system 112 may include two or more separate data processing circuits with one portion of the processing being performed at the manipulator assembly 102, another portion of the processing being performed at the master assembly 106, and/or the like. In some examples, the control system 112 may include other types of processing circuitry, such as application-specific integrated circuits (ASICs) and/or field-programmable gate array (FPGAs). The control system 112 may be implemented using hardware, firmware, software, or a combination thereof.

In some examples, the control system 112 may receive feedback from the medical instrument 104, such as force and/or torque feedback. Responsive to the feedback, the control system 112 may transmit signals to the master assembly 106. In some examples, the control system 112 may transmit signals instructing one or more actuators of the manipulator assembly 102 to move the medical instrument 104. In some examples, the control system 112 may transmit informational displays regarding the feedback to the display system 110 for presentation or perform other types of actions based on the feedback.

The control system 112 may include a virtual visualization system to provide navigation assistance to operator O when controlling the medical instrument 104 during an image-guided medical procedure. Virtual navigation using the virtual visualization system may be based upon an acquired pre-operative or intra-operative dataset of anatomic passageways of the patient P. The control system 112 or a separate computing device may convert the recorded images, using programmed instructions alone or in combination with operator inputs, into a model of the patient anatomy. The model may include a segmented two-dimensional or three-dimensional composite representation of a partial or an entire anatomic organ or anatomic region. An image data set may be associated with the composite representation. The virtual visualization system may obtain sensor data from the sensor system 108 that is used to compute an (e.g., approximate) location of the medical instrument 104 with respect to the anatomy of patient P. The sensor system 108 may be used to register and display the medical instrument 104 together with the pre-operatively or intra-operatively recorded images. For example, PCT Publication WO 2016/191298 (published Dec. 1, 2016 and titled “Systems and Methods of Registration for Image Guided Surgery”), which is incorporated by reference herein in its entirety, discloses example systems.

During a virtual navigation procedure, the sensor system 108 may be used to compute the (e.g., approximate) location of the medical instrument 104 with respect to the anatomy of patient P. The location can be used to produce both macro-level (e.g., external) tracking images of the anatomy of patient P and virtual internal images of the anatomy of patient P. The system may include one or more electromagnetic (EM) sensors, fiber optic sensors, and/or other sensors to register and display a medical instrument together with pre-operatively recorded medical images. For example, U.S. Pat. No. 8,900,131 (filed May 13, 2011 and titled “Medical System Providing Dynamic Registration of a Model of an Anatomic Structure for Image-Guided Surgery”), which is incorporated by reference herein in its entirety, discloses example systems.

Medical system 100 may further include operations and support systems (not shown) such as illumination systems, steering control systems, irrigation systems, and/or suction systems. In some embodiments, the medical system 100 may include more than one manipulator assembly and/or more than one master assembly. The exact number of manipulator assemblies may depend on the medical procedure and space constraints within the procedural room, among other factors. Multiple master assemblies may be co-located or they may be positioned in separate locations. Multiple master assemblies may allow more than one operator to control one or more manipulator assemblies in various combinations.

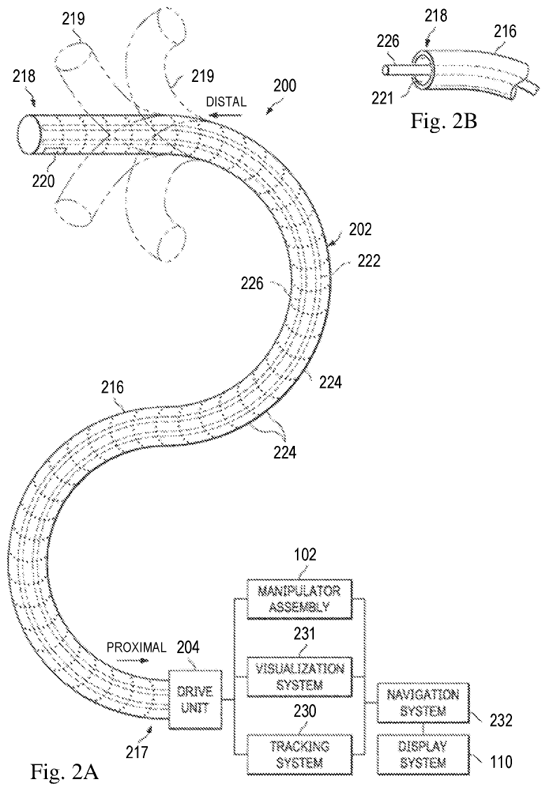

FIG. 2A is a simplified diagram of a medical instrument system 200 according to some embodiments. The medical instrument system 200 includes a flexible elongate device 202 (also referred to as elongate device 202), a drive unit 204, and a medical tool 226 that collectively is an example of a medical instrument 104 of a medical system 100. The medical system 100 may be a teleoperated system, a non-teleoperated system, or a hybrid teleoperated and non-teleoperated system, as explained with reference to FIG. 1. A visualization system 231, tracking system 230, and navigation system 232 are also shown in FIG. 2A and are example components of the control system 112 of the medical system 100. In some examples, the medical instrument system 200 may be used for non-teleoperational exploratory procedures or in procedures involving traditional manually operated medical instruments, such as endoscopy. The medical instrument system 200 may be used to gather (e.g., measure) a set of data points corresponding to locations within anatomic passageways of a patient, such as patient P.

The elongate device 202 is coupled to the drive unit 204. The elongate device 202 includes a channel 221 through which the medical tool 226 may be inserted. The elongate device 202 navigates within patient anatomy to deliver the medical tool 226 to a procedural site. The elongate device 202 includes a flexible body 216 having a proximal end 217 and a distal end 218. In some examples, the flexible body 216 may have an approximately 3 mm outer diameter. Other flexible body outer diameters may be larger or smaller.

Medical instrument system 200 may include the tracking system 230 for determining the position, orientation, speed, velocity, pose, and/or shape of the flexible body 216 at the distal end 218 and/or of one or more segments 224 along flexible body 216, as will be described in further detail below. The tracking system 230 may include one or more sensors and/or imaging devices. The flexible body 216, such as the length between the distal end 218 and the proximal end 217, may include multiple segments 224. The tracking system 230 may be implemented using hardware, firmware, software, or a combination thereof. In some examples, the tracking system 230 is part of control system 112 shown in FIG. 1.

Tracking system 230 may track the distal end 218 and/or one or more of the segments 224 of the flexible body 216 using a shape sensor 222. The shape sensor 222 may include an optical fiber aligned with the flexible body 216 (e.g., provided within an interior channel of the flexibly body 216 or mounted externally along the flexible body 216). In some examples, the optical fiber may have a diameter of approximately 200 μm. In other examples, the diameter may be larger or smaller. The optical fiber of the shape sensor 222 may form a fiber optic bend sensor for determining the shape of flexible body 216. Optical fibers including Fiber Bragg Gratings (FBGs) may be used to provide strain measurements in structures in one or more dimensions. Various systems and methods for monitoring the shape and relative position of an optical fiber in three dimensions, which may be applicable in some embodiments, are described in U.S. Patent Application Publication No. 2006/0013523 (filed Jul. 13, 2005 and titled “Fiber optic position and shape sensing device and method relating thereto”); U.S. Pat. No. 7,772,541 (filed on Mar. 12, 2008 and titled “Fiber Optic Position and/or Shape Sensing Based on Rayleigh Scatter”); and U.S. Pat. No. 8,773,650 (filed on Sep. 2, 2010 and titled “Optical Position and/or Shape Sensing”), which are all incorporated by reference herein in their entireties. Sensors in some embodiments may employ other suitable strain sensing techniques, such as Rayleigh scattering, Raman scattering, Brillouin scattering, and Fluorescence scattering.

In some examples, the shape of the flexible body 216 may be determined using other techniques. For example, a history of the position and/or pose of the distal end 218 of the flexible body 216 can be used to reconstruct the shape of flexible body 216 over an interval of time (e.g., as the flexible body 216 is advanced or retracted within a patient anatomy). In some examples, the tracking system 230 may alternatively and/or additionally track the distal end 218 of the flexible body 216 using a position sensor system 220. Position sensor system 220 may be a component of an EM sensor system with the position sensor system 220 including one or more position sensors. Although the position sensor system 220 is shown as being near the distal end 218 of the flexible body 216 to track the distal end 218, the number and location of the position sensors of the position sensor system 220 may vary to track different regions along the flexible body 216. In one example, the position sensors include conductive coils that may be subjected to an externally generated electromagnetic field. Each coil of position sensor system 220 may produce an induced electrical signal having characteristics that depend on the position and orientation of the coil relative to the externally generated electromagnetic field. The position sensor system 220 may measure one or more position coordinates and/or one or more orientation angles associated with one or more portions of flexible body 216. In some examples, the position sensor system 220 may be configured and positioned to measure six degrees of freedom, e.g., three position coordinates X, Y, Z and three orientation angles indicating pitch, yaw, and roll of a base point. In some examples, the position sensor system 220 may be configured and positioned to measure five degrees of freedom, e.g., three position coordinates X, Y, Z and two orientation angles indicating pitch and yaw of a base point. Further description of a position sensor system, which may be applicable in some embodiments, is provided in U.S. Pat. No. 6,380,732 (filed Aug. 11, 1999 and titled “Six-Degree of Freedom Tracking System Having a Passive Transponder on the Object Being Tracked”), which is incorporated by reference herein in its entirety.

In some embodiments, the tracking system 230 may alternately and/or additionally rely on a collection of pose, position, and/or orientation data stored for a point of an elongate device 202 and/or medical tool 226 captured during one or more cycles of alternating motion, such as breathing. This stored data may be used to develop shape information about the flexible body 216. In some examples, a series of position sensors (not shown), such as EM sensors like the sensors in position sensor 220 or some other type of position sensors may be positioned along the flexible body 216 and used for shape sensing. In some examples, a history of data from one or more of these position sensors taken during a procedure may be used to represent the shape of elongate device 202, particularly if an anatomic passageway is generally static.

FIG. 2B is a simplified diagram of the medical tool 226 within the elongate device 202 according to some embodiments. The flexible body 216 of the elongate device 202 may include the channel 221 sized and shaped to receive the medical tool 226. In some embodiments, the medical tool 226 may be used for procedures such as diagnostics, imaging, surgery, biopsy, ablation, illumination, irrigation, suction, electroporation, etc. Medical tool 226 can be deployed through channel 221 of flexible body 216 and operated at a procedural site within the anatomy. Medical instrument 226 may be, for example, an image capture probe, a biopsy tool (e.g., a needle, grasper, brush, etc.), an ablation tool (e.g., a laser ablation tool, radio frequency (RF) ablation tool, cryoablation tool, thermal ablation tool, heated liquid ablation tool, etc.), an electroporation tool, and/or another surgical, diagnostic, or therapeutic tool. In some examples, the medical tool 226 may include an end effector having a single working member such as a scalpel, a blunt blade, an optical fiber, an electrode, and/or the like. Other end types of end effectors may include, for example, forceps, graspers, scissors, staplers, clip appliers, and/or the like. Other end effectors may further include electrically activated end effectors such as electrosurgical electrodes, transducers, sensors, and/or the like.

The medical tool 226 may be a biopsy tool used to remove sample tissue or a sampling of cells from a target anatomic location. In some examples, the biopsy tool is a flexible needle. The biopsy tool may further include a sheath that can surround the flexible needle to protect the needle and interior surface of the channel 221 when the biopsy tool is within the channel 221. The medical tool 226 may be an image capture probe that includes a distal portion with a stereoscopic or monoscopic camera that may be placed at or near the distal end 218 of flexible body 216 for capturing images (e.g., still or video images). The captured images may be processed by the visualization system 231 for display and/or provided to the tracking system 230 to support tracking of the distal end 218 of the flexible body 216 and/or one or more of the segments 224 of the flexible body 216. The image capture probe may include a cable for transmitting the captured image data that is coupled to an imaging device at the distal portion of the image capture probe. In some examples, the image capture probe may include a fiber-optic bundle, such as a fiberscope, that couples to a more proximal imaging device of the visualization system 231. The image capture probe may be single-spectral or multi-spectral, for example, capturing image data in one or more of the visible, near-infrared, infrared, and/or ultraviolet spectrums. The image capture probe may also include one or more light emitters that provide illumination to facilitate image capture. In some examples, the image capture probe may use ultrasound, x-ray, fluoroscopy, CT, MRI, or other types of imaging technology.

In some examples, the image capture probe is inserted within the flexible body 216 of the elongate device 202 to facilitate visual navigation of the elongate device 202 to a procedural site and then is replaced within the flexible body 216 with another type of medical tool 226 that performs the procedure. In some examples, the image capture probe may be within the flexible body 216 of the elongate device 202 along with another type of medical tool 226 to facilitate simultaneous image capture and tissue intervention, such as within the same channel 221 or in separate channels. A medical tool 226 may be advanced from the opening of the channel 221 to perform the procedure (or some other functionality) and then retracted back into the channel 221 when the procedure is complete. The medical tool 226 may be removed from the proximal end 217 of the flexible body 216 or from another optional instrument port (not shown) along flexible body 216.

In some examples, the elongate device 202 may include integrated imaging capability rather than utilize a removable image capture probe. For example, the imaging device (or fiber-optic bundle) and the light emitters may be located at the distal end 218 of the elongate device 202. The flexible body 216 may include one or more dedicated channels that carry the cable(s) and/or optical fiber(s) between the distal end 218 and the visualization system 231. Here, the medical instrument system 200 can perform simultaneous imaging and tool operations.

In some examples, the medical tool 226 is capable of controllable articulation. The medical tool 226 may house cables (which may also be referred to as pull wires), linkages, or other actuation controls (not shown) that extend between its proximal and distal ends to controllably bend the distal end of medical tool 226, such as discussed herein for the flexible elongate device 202. The medical tool 226 may be coupled to a drive unit 204 and the manipulator assembly 102. In these examples, the elongate device 202 may be excluded from the medical instrument system 200 or may be a flexible device that does not have controllable articulation. Steerable instruments or tools, applicable in some embodiments, are further described in detail in U.S. Pat. No. 7,316,681 (filed on Oct. 4, 2005 and titled “Articulated Surgical Instrument for Performing Minimally Invasive Surgery with Enhanced Dexterity and Sensitivity”) and U.S. Pat. No. 9,259,274 (filed Sept. 30, 2008 and titled “Passive Preload and Capstan Drive for Surgical Instruments”), which are incorporated by reference herein in their entireties.

The flexible body 216 of the elongate device 202 may also or alternatively house cables, linkages, or other steering controls (not shown) that extend between the drive unit 204 and the distal end 218 to controllably bend the distal end 218 as shown, for example, by broken dashed line depictions 219 of the distal end 218 in FIG. 2A. In some examples, at least four cables are used to provide independent up-down steering to control a pitch of the distal end 218 and left-right steering to control a yaw of the distal end 218. In these examples, the flexible elongate device 202 may be a steerable catheter. Examples of steerable catheters, applicable in some embodiments, are described in detail in PCT Publication WO 2019/018736 (published Jan. 24, 2019 and titled “Flexible Elongate Device Systems and Methods”), which is incorporated by reference herein in its entirety.

In embodiments where the elongate device 202 and/or medical tool 226 are actuated by a teleoperational assembly (e.g., the manipulator assembly 102), the drive unit 204 may include drive inputs that removably couple to and receive power from drive elements, such as actuators, of the teleoperational assembly. In some examples, the elongate device 202 and/or medical tool 226 may include gripping features, manual actuators, or other components for manually controlling the motion of the elongate device 202 and/or medical tool 226. The elongate device 202 may be steerable or, alternatively, the elongate device 202 may be non-steerable with no integrated mechanism for operator control of the bending of distal end 218. In some examples, one or more channels 221 (which may also be referred to as lumens), through which medical tools 226 can be deployed and used at a target anatomical location, may be defined by the interior walls of the flexible body 216 of the elongate device 202.

In some examples, the medical instrument system 200 (e.g., the elongate device 202 or medical tool 226) may include a flexible bronchial instrument, such as a bronchoscope or bronchial catheter, for use in examination, diagnosis, biopsy, and/or treatment of a lung. The medical instrument system 200 may also be suited for navigation and treatment of other tissues, via natural or surgically created connected passageways, in any of a variety of anatomic systems, including the colon, the intestines, the kidneys and kidney calices, the brain, the heart, the circulatory system including vasculature, and/or the like.

The information from the tracking system 230 may be sent to the navigation system 232, where the information may be combined with information from the visualization system 231 and/or pre-operatively obtained models to provide the physician, clinician, surgeon, or other operator with real-time position information. In some examples, the real-time position information may be displayed on the display system 110 for use in the control of the medical instrument system 200. In some examples, the navigation system 232 may utilize the position information as feedback for positioning medical instrument system 200. Various systems for using fiber optic sensors to register and display a surgical instrument with surgical images, applicable in some embodiments, are provided in U.S. Pat. No. 8,900,131 (filed May 13, 2011 and titled “Medical System Providing Dynamic Registration of a Model of an Anatomic Structure for Image-Guided Surgery”), which is incorporated by reference herein in its entirety.

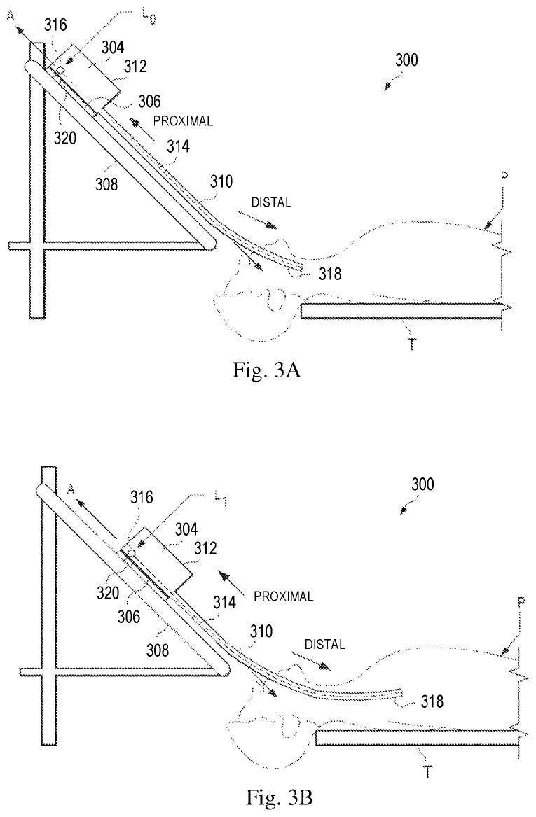

FIGS. 3A and 3B are simplified diagrams of side views of a patient coordinate space including a medical instrument mounted on an insertion assembly according to some embodiments. As shown in FIGS. 3A and 3B, a surgical environment 300 may include a patient P positioned on the patient table T. Patient P may be stationary within the surgical environment 300 in the sense that gross patient movement is limited by sedation, restraint, and/or other means. Cyclic anatomic motion, including respiration and cardiac motion, of patient P may continue. Within surgical environment 300, a medical instrument 304 is used to perform a medical procedure which may include, for example, surgery, biopsy, ablation, illumination, irrigation, suction, or electroporation. The medical instrument 304 may also be used to perform other types of procedures, such as a registration procedure to associate the position, orientation, and/or pose data captured by the sensor system 108 to a desired (e.g., anatomical or system) reference frame. The medical instrument 304 may be, for example, the medical instrument 104. In some examples, the medical instrument 304 may include an elongate device 310 (e.g., a catheter) coupled to an instrument body 312. Elongate device 310 includes one or more channels sized and shaped to receive a medical tool.

Elongate device 310 may also include one or more sensors (e.g., components of the sensor system 108). In some examples, a shape sensor 314 may be fixed at a proximal point 316 on the instrument body 312. The proximal point 316 of the shape sensor 314 may be movable with the instrument body 312, and the location of the proximal point 316 with respect to a desired reference frame may be known (e.g., via a tracking sensor or other tracking device). The shape sensor 314 may measure a shape from the proximal point 316 to another point, such as a distal end 318 of the elongate device 310. The shape sensor 314 may be aligned with the elongate device 310 (e.g., provided within an interior channel or mounted externally). In some examples, the shape sensor 314 may optical fibers used to generate shape information for the elongate device 310.

In some examples, position sensors (e.g., EM sensors) may be incorporated into the medical instrument 304. A series of position sensors may be positioned along the flexible elongate device 310 and used for shape sensing. Position sensors may be used alternatively to the shape sensor 314 or with the shape sensor 314, such as to improve the accuracy of shape sensing or to verify shape information.

Elongate device 310 may house cables, linkages, or other steering controls that extend between the instrument body 312 and the distal end 318 to controllably bend the distal end 318. In some examples, at least four cables are used to provide independent up-down steering to control a pitch of distal end 318 and left-right steering to control a yaw of distal end 318. The instrument body 312 may include drive inputs that removably couple to and receive power from drive elements, such as actuators, of a manipulator assembly.

The instrument body 312 may be coupled to an instrument carriage 306. The instrument carriage 306 may be mounted to an insertion stage 308 that is fixed within the surgical environment 300. Alternatively, the insertion stage 308 may be movable but have a known location (e.g., via a tracking sensor or other tracking device) within surgical environment 300. Instrument carriage 306 may be a component of a manipulator assembly (e.g., manipulator assembly 102) that couples to the medical instrument 304 to control insertion motion (e.g., motion along an insertion axis A) and/or motion of the distal end 318 of the elongate device 310 in multiple directions, such as yaw, pitch, and/or roll. The instrument carriage 306 or insertion stage 308 may include actuators, such as servomotors, that control motion of instrument carriage 306 along the insertion stage 308.

A sensor device 320, which may be a component of the sensor system 108, may provide information about the position of the instrument body 312 as it moves relative to the insertion stage 308 along the insertion axis A. The sensor device 320 may include one or more resolvers, encoders, potentiometers, and/or other sensors that measure the rotation and/or orientation of the actuators controlling the motion of the instrument carriage 306, thus indicating the motion of the instrument body 312. In some embodiments, the insertion stage 308 has a linear track as shown in FIGS. 3A and 3B. In some embodiments, the insertion stage 308 may have curved track or have a combination of curved and linear track sections.

FIG. 3A shows the instrument body 312 and the instrument carriage 306 in a retracted position along the insertion stage 308. In this retracted position, the proximal point 316 is at a position L0 on the insertion axis A. The location of the proximal point 316 may be set to a zero value and/or other reference value to provide a base reference (e.g., corresponding to the origin of a desired reference frame) to describe the position of the instrument carriage 306 along the insertion stage 308. In the retracted position, the distal end 318 of the elongate device 310 may be positioned just inside an entry orifice of patient P. Also in the retracted position, the data captured by the sensor device 320 may be set to a zero value and/or other reference value (e.g., I=0). In FIG. 3B, the instrument body 312 and the instrument carriage 306 have advanced along the linear track of insertion stage 308, and the distal end 318 of the elongate device 310 has advanced into patient P. In this advanced position, the proximal point 316 is at a position L1 on the insertion axis A. In some examples, the rotation and/or orientation of the actuators measured by the sensor device 320 indicating movement of the instrument carriage 306 along the insertion stage 308 and/or one or more position sensors associated with instrument carriage 306 and/or the insertion stage 308 may be used to determine the position L1 of the proximal point 316 relative to the position L0. In some examples, the position L1 may further be used as an indicator of the distance or insertion depth to which the distal end 318 of the elongate device 310 is inserted into the passageway(s) of the anatomy of patient P.

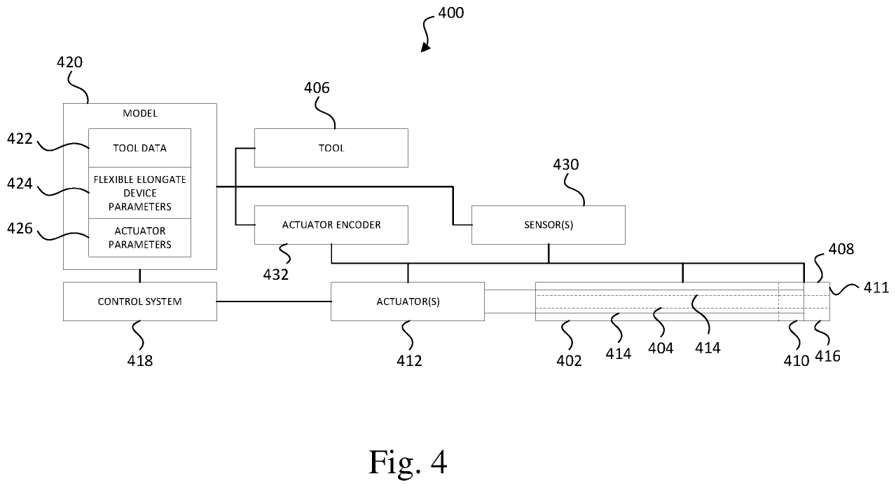

FIG. 4 is a simplified diagram of a medical instrument system 400 including a flexible elongate device 402 according to some embodiments. According to some embodiments consistent with FIGS. 1-3, the medical instrument system 400 may correspond to the medical instrument system 200 and/or the flexible elongate device 402 may correspond to the elongate device 202.

The flexible elongate device 402 can include a flexible body and a main lumen 404 that extends through the flexible body. The main lumen 404 may provide a delivery channel for a medical tool 406, such as a vision probe, a biopsy tool (e.g., a needle, brush, cryoprobe, or forceps), an ablation tool, an electroporation tool, an ultrasound device (e.g., endobronchial ultrasound (EBUS) probe), a chemical delivery tool, and/or the like, to be inserted through the flexible body of the flexible elongate device 402.

As shown in FIG. 4, the flexible body of the flexible elongate device 402 can include an articulable body portion 408, which may be in a distal section 410 including a distal tip 411 thereof. In the illustrated embodiment, the system 400 includes one or more actuators 412 that control articulation of the articulable body portion 408 via manipulation of one or more control elements 414, such as pull wires, tendons, push rods, and/or the like, connected to a control structure 416 of the articulable body portion 408. Operation of the actuator 412 causes the respective control element 414 to pull back to cause the articulable body portion 408 to bend in the direction of the control element 414 or release allowing the articulable body portion 408 to return to a straighter configuration.

As discussed above, when the tool 406 is inserted within the lumen 404 of the flexible elongate device 402, the control response of the (e.g., distal) articulable body portion 408 of the flexible elongate device 402 may change in response to the tool insertion. For example, insertion of the tool 406 may change the combined stiffness, as well as other physical properties that impact articulation control, of the flexible elongate device 402 and tool 406. With the added physical properties of the tool 406, the articulable body portion 408 may be deflected from an expected position as the tool 406 imparts additional force on the tension of the control element 414. It will be understood that the expected position, as discussed herein, may include a position of the articulable body portion 408 prior to deflection and may also include an intended position that was desired/commanded, but not reached due to the control response being different due to insertion of the tool and the impact on the system 400.

The systems and methods disclosed herein advantageously reduce deviation of the articulable body portion 408 of the flexible elongate device 402 that may result when the tool 406 is inserted into the lumen 404 of the flexible elongate device 402. The position of the articulable body portion 408 of the flexible elongate device 402 is determined using a model-based approach, where the model includes tool properties (e.g., in addition or alternative to a direct measurement approach), and then used to control articulation of the flexible elongate device 402.

The system 400 includes in a control system 418 that operates or accesses a model 420 to control operation of the actuator(s) 412 to thereby control position of the articulable body portion 408 of the flexible elongate device 402. The control system 418 is configured to: determine an expected position (e.g., a position commanded by a user operating the system 400), of the articulable body portion 408 of the flexible elongate device 402, determine tool data or parameters 422 associated with the tool 406, determine adjustment data based on the model 420 of the flexible elongate device 402 with the tool data 422 to cause an actual position of the articulable body portion 408 to correspond with the expected position when the tool 406 is inserted within the lumen 404, and operate the actuator(s) 412 to control articulation the articulable body portion 408 of the flexible elongate device 402 according to the adjustment data.

The tool data 422 may include a tool type and one or more properties of the tool 406. The tool type can be received at the control system 418 from a user input or determined using one or more sensors. Each tool type may be associated with one or more properties, such as in a database. In another example, the properties of the tool may be received from user input or determined using one or more sensors. The properties of the tool 406 may include one or more of: a stiffness of the tool 406, an inertia of the tool 406, and/or a friction of the tool 406. Other types of properties that may impact the articulation response when tools are inserted within the flexible elongate device 402 may also be used.

In some examples, the model 420 may account for parameters or properties 424 of the flexible elongate device 402 in combination with the parameters 422 of the tool 406. For example, the combined parameters can include a combined stiffness, combined friction, and/or combined inertia. For example, the combined stiffness of the tool 406 and the articulable body portion 408 requires a greater amount of force (e.g., torque generated by the actuator(s) 412) to bend or otherwise manipulate the position of the articulable body portion 408. In another example, due to the increased mass when the tool 406 is combined with the articulable body portion 408, the resulting combined inertia requires a greater amount of force (e.g., torque generated by the actuator(s) 412) to begin and stop movement of the articulable body portion 408, relative to movement of the articulable body portion 408 alone. In another example, when the tool 406 is inserted within the lumen 404 of the flexible elongate device 402, a greater amount of force to bend or otherwise manipulate the position of the articulable body portion 408 is required to overcome the increased friction caused by the surface contact of the tool 406 and the inner lumen 404.

As will be understood, different tools 406 that are inserted within the flexible elongate device 402 may change the combined properties of the flexible elongate device 402 and tool 406. For example, different types of tools 406 may have different stiffnesses, resulting in a different overall stiffness for the flexible elongate device 402 and tool 406 system. As such, the model 420 can be updated based on the one or more properties of the tool 406 inserted within the flexible elongate device 402. In yet further examples that utilize actuator torque as an input, the model 420 may also account for parameters or properties 426 of the actuator(s) 412 in combination with the parameters 422, 424 of the tool 406 and/or the flexible elongate device 402. For example, the combined parameters can further include actuator stiffness, actuator inertia, and/or actuator friction. In some examples, the model defines a relationship between the articulation of the articulable body portion 408 as a function of the operation of the actuator(s) 412 and the properties of the tool. The model may also define the relative impact of different model properties to the relationship between the articulation of the articulable body portion 408 and the operation of the actuator(s) 412.

As discussed above, the control system may be configured to determine adjustment data for operating the actuator to cause a deviated position of the articulable body portion to correspond with the expected position based on the model, which may diverge from one another due to the insertion of a tool into the flexible elongate device and/or removal of a tool from the flexible elongate device.

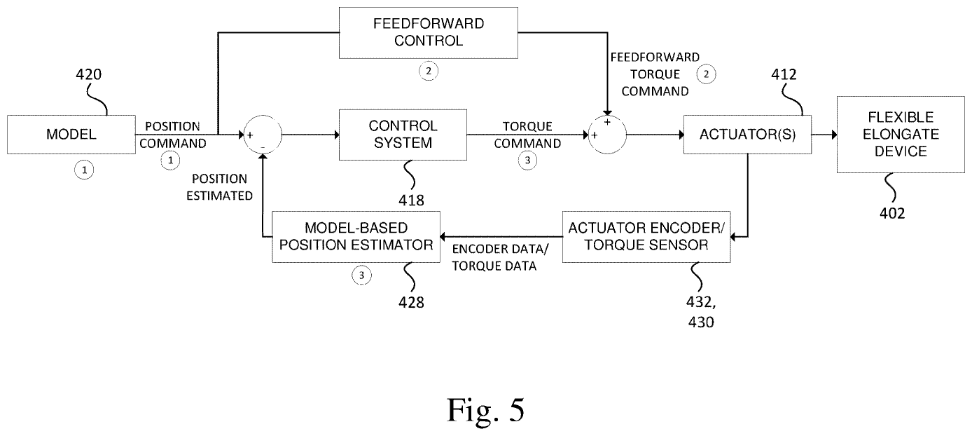

As shown in FIG. 5, the adjustment data determined by the model 420 can take a variety of forms depending on the desired algorithm. For example, the adjustment data may be: (1) a position command value to account for the deviated position (e.g., include an error value) of the articulable body portion 408; (2) a feedforward control value (e.g., a torque value) determined using the model 420 to account for the deviated position of the articulable body portion 408; or (3) a control value (e.g., a torque value) from a feedback controller/estimator 428 based on an error of the expected position of the articulable body portion 408 and the estimated position using the updated model 420.

In other words, in adjustment data (1), the model 420 may be used to determine a new commanded position for the articulable body portion 408 to account for the divergence resulting from tool insertion/removal. In this example, the model 420 may add a correction/error value to a desired position input by a user. Thereafter, the system 400 may operate according to the position with the correction/error value to control the actuator(s) 412 accordingly, such that the articulable body portion 408 is manipulated to the desired position. In adjustment data (2), the model 420 may provide feedforward control to adjust parameters of the model 420 (e.g., a stiffness of tool 406 and flexible elongate device 402 and/or other parameters, as discussed below) into torque values for the actuator(s) 412 to account for the divergence of the articulable body portion 408 resulting from tool insertion/removal. In this example, the model 420 proactively impacts actuator control to increase or decrease the torque output by the actuator(s) 412 for manipulation of the articulable body portion 408 to a desired position input by a user to account for divergence due to the tool insertion/removal. In adjustment data (3), a model-based estimator 428 may estimate the position of the articulable body portion 408, e.g., a bending angle, where the model parameters in the estimator 428 are updated based on the tool data 422. In this example, data from the actuator(s) 412 (e.g., encoder data or torque data) is input to the model 420 and the model 420 uses the data and the tool data to determine an estimated position accounting for the deviation of the articulable body portion 408 resulting from insertion/removal of the tool 406. With the error between the desired and estimated positions, the control system 418 then outputs a torque value to correct for the estimated deviation of the articulable body portion 408. The torque values in adjustment data (2) and (3) are used to command the actuator(s) 412 of the system 400, which results in movement control of the articulable body portion 408 of the flexible elongate device 402.

As shown in FIG. 4, the system 400 may include one or more sensors 430 that are configured to determine the actual position of the articulable body portion 408 of the flexible elongate device 402. For example, the sensor(s) 430 may measure or indicate a shape, position(s), and/or bend angle of the articulable body portion 408. The sensor(s) 430 can take any suitable form, including, for example, a shape sensor (e.g., fiber), a position sensor (e.g., an electro-magnetic sensor), and/or an imaging sensor (e.g., camera, ultrasound, fluoroscope, etc.). In embodiments that utilize actuator torque as an input, the sensor(s) 430 may also include a current sensor configured to measure a current through a motor of the actuator(s) 412, which is then converted to motor torque, and/or a torque sensor coupled to an output of a gearbox of or coupled to the actuator(s) 412.

In some forms, the sensor(s) 430 may provide continuous or periodic data to the control system 418 regarding the state (e.g., position, bending angle, shape, etc.) of the articulable body portion 408, including, for example, as the flexible elongate device 402 traverses anatomy of a patient. This data may be input into the model 420 to update the model, including the relationship between the position of the articulable body portion 408 and operation of the actuator(s) 412, tool parameters, etc.

In one embodiment, the expected position for the articulable body portion 408 may be a position for performing a medical procedure using the tool 406, the actual position may be a deviation from the expected position caused by movement of the tool 406 within the lumen 404 of the flexible elongate device 402, and the control system 418 may be configured to operate the actuator(s) 412 to control articulation the articulable body portion 408 of the flexible elongate device 402 to return the distal tip 411 of the flexible elongate device 402 to the expected position.

In a first approach, the model 420 may define the relationship between movement of the actuator(s) 412 (e.g., encoder data) and articulation of the articulable body portion 408 of the flexible elongate device 402 (e.g., a position, shape, and/or bending angle of the articulable body portion 408). In this approach, encoder data is utilized from one or more actuator encoders 432 for the actuator(s) 412 located proximal to the flexible elongate device 402 that control the articulation of the articulable body portion 408 of the flexible elongate device 402. The model 420 correlates the encoder data with the expected position of the articulable body portion 408. The actuator encoders 432 are configured to generate encoder data regarding operational states (e.g., motor position, speed, etc.) of the actuator(s) 412.

The model 420 may be calibrated by continuously updating the model 420 in real-time as the flexible elongate device 402 traverses anatomical passageways of a patient. The data (e.g., shape data, position data, etc.) from the sensor(s) 430 provides the actual position (e.g., ground truth) of the articulable body portion 408. The associated encoder data from the actuator encoder(s) 432 may be used to update the parameters of the model 420 and, in particular, the relationships between the encoder data and expected positions of the articulable body portion 408. Further, when a tool 406 is inserted into or removed from the flexible elongate device 402, the latest real-time model 420 may be updated accordingly to account for the effects that the particular tool insertion/removal imparts on the flexible elongate device 402. The real-time updating of the model 420 accounts for any effects of tool properties, hysteresis, and anatomical environment, for example. Hysteresis refers to the dependence of a given state of a system on its history, e.g., the system believing that the articulable body portion 408 is in the expected position after insertion of the tool 406. Further, in some instances, the route of the flexible elongate device 402 through a given anatomical environment (e.g., straight, circuitous, acuteness of bends, etc.) may impact the parameters of the model 420. Then, if the sensor 430 has an error or otherwise isn't available (e.g., failure of the shape sensor, lack of a shape sensor, etc.), the latest updated model 420 can be used with the newest encoder data from the actuator encoder(s) 432 to correct any deviations of the articulable body portion 408 due to movement of the tool 406.

Alternatively, the model 420 may be generated for the flexible elongate device 402 prior to a procedure and not be updated in real-time for calibration during traversal of the anatomical pathways. Rather, the model 420 may be only updated when a tool 406 is inserted and/or removed. In this case, the relationships between the encoder data and expected positions of the articulable body portion 408 resulting from the insertion/removal of the tool 406 may be utilized to update the model 420. Then, if the sensor 430 has an error or otherwise isn't available (e.g., failure of the shape sensor, lack of a shape sensor, etc.), the latest updated model 420 can be used with the newest encoder data to correct any deviations of the articulable body portion 408 due to movement of the tool 406.

In a second approach, the model 420 may define the relationship between a torque (e.g., motor torque) of the actuator(s) 412 located proximal to the flexible elongate device 402 that control articulation of the articulable body portion 408 of the flexible elongate device 402 and the articulation of the articulable body portion 408 (e.g., a bending angle of the articulable body portion 408). As discussed above, the parameters of the model 420 may include parameters 424 of the flexible elongate device 402, such as stiffness, inertia, friction, damping, etc., and may also include dynamic properties 426 of the actuator(s) 412, such as actuator inertia, friction, stiffness, etc.

The models 420 may be calibrated by continuously updating the model 420 in real-time as the flexible elongate device 402 traverses anatomical passageways of a patient. The data (e.g., shape data) from the sensor(s) 430 provides the actual position (e.g., ground truth) of the articulable body portion 408. The sensor data may be used to update the parameters of the model 420 and, in particular, the relationships between the position of the articulable body portion 408 (e.g., bending angle) and the actuator torque. Further, when a tool 406 is inserted into or removed from the flexible elongate device 402, the latest real-time model 420 may be updated accordingly to account for the effects that the particular tool insertion/removal imparts on the flexible elongate device 402. The real-time updating of the model 420 accounts for any effects of tool properties, hysteresis, and anatomical environment, for example. Then, if the sensor 430 has an error or otherwise isn't available (e.g., failure of the shape sensor, lack of a shape sensor, etc.), the latest updated model 420 can be used with the newest actuator torque to correct any deviations of the articulable body portion 408 due to movement of the tool 406.

Alternatively, the model 420 may be generated for the flexible elongate device 402 prior to a procedure and not be updated in real-time for calibration during traversal of the anatomical pathways. Rather, the model 420 may be only updated when a tool 406 is inserted and/or removed. In this case, the relationships between the actuator torque and expected positions of the articulable body portion 408 resulting from the insertion/removal of the tool 406 may be utilized to update the model 420. Then, if the sensor 430 has an error or otherwise isn't available (e.g., failure of the shape sensor, lack of a shape sensor, etc.), the latest updated model 420 can be used with the newest actuator torque to correct any deviations of the articulable body portion 408 due to movement of the tool 406.

If desired, with any of the above approaches and even in situations where the shape sensor 430 is available and functioning properly, the system 400 may utilized the (2) feedforward control to adjust parameters of the model 420 (e.g., a stiffness of tool 406 and flexible elongate device 402 and/or other parameters, as discussed below) into torque values for the actuator(s) 412 to account for the divergence of the articulable body portion 408 resulting from tool insertion/removal. The feedforward control may allow the control system 418 to more quickly react to and correct any position deviations of the articulable body portion 408.

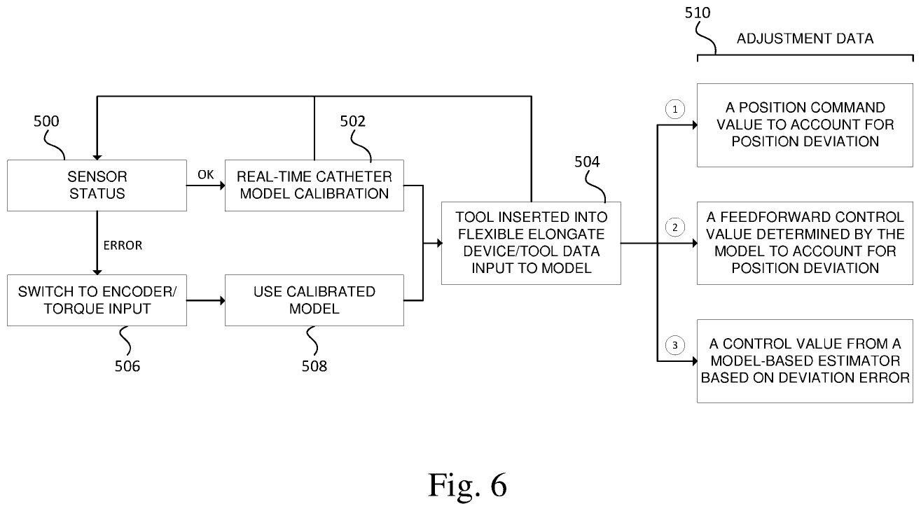

FIG. 6 illustrates one example flow chart for operation of a medical system (e.g., medical system 400) utilizing a model (e.g., model 420) to account for deviation of an articulable body portion of a flexible elongate device (e.g., articulable body portion 408 and flexible elongate device 402) due to insertion of a tool (e.g., tool 406) into the flexible elongate device according to some embodiments. The flowchart is illustrated as a set of operations or processes 500 through 510. Not all of the illustrated processes may be performed in all embodiments of the flowchart. Additionally, one or more processes that are not expressly illustrated in FIG. 6 may be included before, after, in between, or as part of the processes 500 through 510. Processes may also be performed in different orders. In some embodiments, one or more of the processes 500 through 510 may be implemented, at least in part, in the form of executable code stored on non-transitory, tangible, machine-readable media that when run by one or more processors (e.g., the processors of a controller) may cause the one or more processors to perform one or more of the processes. In one or more embodiments, the processes 500 through 510 may be performed by a controller.

In process 500, the status of a sensor (e.g., sensor 430) of the flexible elongate device, such as a shape sensor or the like, is determined. If the sensor is operating normally, in process 502, the model is calibrated with data from the sensor. At process 504, a tool is inserted into the flexible elongate device and tool data (e.g., tool data 422) associated with the tool is input to the model. As discussed above, combined data (e.g., flexible elongate device and/or actuator) may also be input to the model. The calibrated model with or without the tool data may be fed back to process 500 of the flowchart.

If the sensor has an error or is otherwise unavailable, in process 506, input is switched to actuator encoder data or actuator torque for the medical system and, in process 508, the actuator encoder data/actuator torque is utilized in the calibrated model. Thereafter, when a tool is inserted in process 504, at process 510, the calibrated model may be utilized to determine adjustment data to account for position deviation of the articulable body portion of the flexible elongate device resulting from insertion of the tool. As discussed above, the adjustment data may be: (1) a position command value to account for the deviated position (e.g., include an error value) of the articulable body portion 408; (2) a feedforward control value (e.g., a torque value) determined using the model 420 to account for the deviated position of the articulable body portion 408; or (3) a control value (e.g., a torque value) from a feedback controller/estimator 428 based on an error of the expected position of the articulable body portion 408 and the estimated position using the updated model 420.

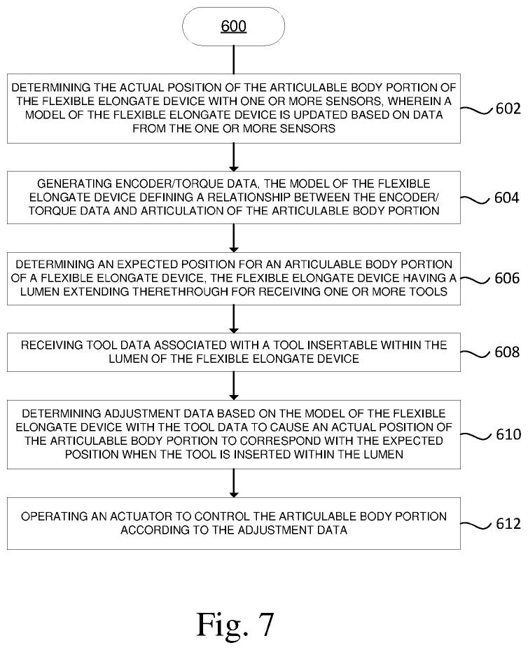

FIG. 7 illustrates a method 600 for operation of a medical system including a flexible elongate device (e.g., the medical system 400 and flexible elongate device 402) according to some embodiments. The method 600 is illustrated as a set of operations or processes 602 through 612. Not all of the illustrated processes may be performed in all embodiments of the method 600. Additionally, one or more processes that are not expressly illustrated in FIG. 7 may be included before, after, in between, or as part of the processes 602 through 612. Processes may also be performed in different orders. In some embodiments, one or more of the processes 602 through 612 may be implemented, at least in part, in the form of executable code stored on non-transitory, tangible, machine-readable media that when run by one or more processors (e.g., the processors of a controller) may cause the one or more processors to perform one or more of the processes. In one or more embodiments, the processes 602 through 612 may be performed by a controller.

In process 602, an actual position of an articulable body portion of a flexible elongate device (e.g., articulable body portion 408 of flexible elongate device 402) is determined with one or more sensors (e.g., sensor(s) 430), so that a model (e.g., model 420) of the flexible elongate device can be updated (or generated when no existing model is available) based on data from the sensors. The model can be updated, for example, based on data from the one or more sensors to adjust the relationships between the encoder data and expected position of the articulable body portion of the flexible elongate device, based on data from the one or more sensors to adjust the relationships between the torque of the actuator and expected position of the articulable body portion of the flexible elongate device, and/or based on data from the one or more sensors as the flexible elongate device traverses anatomy. In process 604, data is generated that is utilized by the model to define a relationship between the data and the articulation of the articulable body portion of the flexible elongate device. In one example, the data can be encoder data regarding operational states of an actuator (e.g., actuator 512) operable to control the articulable body portion generated by an actuator encoder (e.g., actuator encoder 432) and the model can define a relationship between the encoder data and articulation of the articulable body portion caused by the operation of the actuator. In another example, the data can be torque data associated with operation of the actuator and the model can define a relationship between the torque data and articulation of the articulable body portion caused by operation of the actuator. In processes 602 and 604, a model is updated dynamically based on data from one or more sensors while the flexible elongate device traverses an anatomy. This allows for the model to account for factors that affect control response, such as the shape of the flexible elongate device, insertion depth of the flexible elongate device within the anatomy, shape of the anatomy being traversed, hysteresis, etc. If a tool is inserted within the flexible elongate device, the tool properties of the model may also be updated in processes 602 and 604 based on the data from the one or more sensors. In other examples, such as when a suitable sensor is not available or becomes inoperable, the model is not updated during traversal of the anatomy may be used.

At process 606, an expected position for an articulable body portion of a flexible elongate device is determined. The flexible elongate device has a lumen (e.g., lumen 404) extending therethrough for receiving one or more tools. At process 608, tool data is determined associated with a tool (e.g., tool 406) insertable within the lumen of the flexible elongate device. It will be understood that processes 606 and 608 may be interchangeable based on the expected position. For example, the expected position may include a position of the articulable body portion 408 prior to deflection, such that process 606 is performed before process 608. In another example, the expected position may include an intended position that was desired/commanded, but not reached due to the control response being different due to insertion of the tool and the impact on the system, in which case process 606 is performed after process 608.

At process 610, adjustment data based on the model of the flexible elongate with the tool data is determined to cause an actual position of the articulable body portion to correspond with the expected position when the tool is inserted within the lumen. The adjustment data can be, for example, a position command value to account for the deviated position of the articulable body portion, a feedforward control value determined using the model to account for the deviated position, or a control value from a feedback controller based on an error of the expected position and the deviated position using the model. At process 612, the actuator is operated to control articulation of the articulable body portion according to the adjustment data.

One or more components of the embodiments discussed in this disclosure, such as control system 112, 418, may be implemented in software for execution on one or more processors of a computer system. The software may include code that when executed by the one or more processors, configures the one or more processors to perform various functionalities as discussed herein. The code may be stored in a non-transitory computer readable storage medium (e.g., a memory, magnetic storage, optical storage, solid-state storage, etc.). The computer readable storage medium may be part of a computer readable storage device, such as an electronic circuit, a semiconductor device, a semiconductor memory device, a read only memory (ROM), a flash memory, an erasable programmable read only memory (EPROM); a floppy diskette, a CD-ROM, an optical disk, a hard disk, or other storage device. The code may be downloaded via computer networks such as the Internet, Intranet, etc. for storage on the computer readable storage medium. The code may be executed by any of a wide variety of centralized or distributed data processing architectures. The programmed instructions of the code may be implemented as a number of separate programs or subroutines, or they may be integrated into a number of other aspects of the systems described herein. The components of the computing systems discussed herein may be connected using wired and/or wireless connections. In some examples, the wireless connections may use wireless communication protocols such as Bluetooth, near-field communication (NFC), Infrared Data Association (IrDA), home radio frequency (HomeRF), IEEE 802.11, Digital Enhanced Cordless Telecommunications (DECT), and wireless medical telemetry service (WMTS).

Various general-purpose computer systems may be used to perform one or more processes, methods, or functionalities described herein. Additionally or alternatively, various specialized computer systems may be used to perform one or more processes, methods, or functionalities described herein. In addition, a variety of programming languages may be used to implement one or more of the processes, methods, or functionalities described herein.

While certain embodiments and examples have been described above and shown in the accompanying drawings, it is to be understood that such embodiments and examples are merely illustrative and are not limited to the specific constructions and arrangements shown and described, since various other alternatives, modifications, and equivalents will be appreciated by those with ordinary skill in the art.

1. A medical system, comprising:
a flexible elongate device having a lumen extending therethrough for receiving one or more tools and having an articulable body portion;
an actuator configured to control articulation of the articulable body portion of the flexible elongate device;
a control system configured to:
determine an expected position of the articulable body portion of the flexible elongate device;
determine tool data associated with a tool insertable within the lumen of the flexible elongate device;
determine adjustment data based on a model of the flexible elongate device with the tool data to cause an actual position of the articulable body portion of the flexible elongate device to correspond with the expected position when the tool is inserted within the lumen; and
operate the actuator to control articulation of the articulable body portion of the flexible elongate device according to the adjustment data.

2. The medical system of claim 1, wherein the model defines a relationship between operation of the actuator and articulation of the articulable body portion of the flexible elongate device caused by the operation of the actuator.

3. The medical system of claim 1, further comprising an actuator encoder for the actuator, the actuator encoder configured to generate encoder data regarding operational states of the actuator, wherein the model defines a relationship between the encoder data and articulation of the articulable body portion of the flexible elongate device caused by the operation of the actuator.

4. The medical system of claim 3, further comprising one or more sensors configured to determine the actual position of the articulable body portion of the flexible elongate device, wherein the model is updated based on data from the one or more sensors to adjust the relationships between the encoder data and expected position of the articulable body portion of the flexible elongate device.

5. The medical system of claim 4, wherein the control system is configured to utilize the model in combination with the tool data and a variable to account for deviation of the articulable body portion of the flexible elongate device in response to insertion or removal of the tool after receiving an error from one of the one or more sensors, wherein the variable is one of encoder data or torque of the actuator.

6. The medical system of claim 1, wherein the model defines a relationship between torque of the actuator and articulation of the articulable body portion of the flexible elongate device caused by the operation of the actuator.

7. The medical system of claim 6, further comprising one or more sensors configured to determine the actual position of the articulable body portion of the flexible elongate device, wherein the model is updated based on data from the one or more sensors to adjust the relationships between the torque of the actuator and expected position of the articulable body portion of the flexible elongate device.

8. The medical system of claim 7, wherein the control system is configured to utilize the model in combination with the tool data and a variable to account for deviation of the articulable body portion of the flexible elongate device in response to insertion or removal of the tool after receiving an error from one of the one or more sensors, wherein the variable is one of encoder data or torque of the actuator.

9. The medical system of claim 1, wherein the adjustment data comprises a position command value to account for the deviated position of the articulable body portion of the flexible elongate device or a feedforward control value determined using the model to account for the deviated position.

10. (canceled)

11. The medical system of claim 1, wherein the adjustment data comprises a control value from a feedback controller based on an error of the expected position and the deviated position using the model.

12. The medical system of claim 1, wherein the tool data includes a property of the tool; and the model further includes parameters of the actuator.

13. (canceled)

14. The medical system of claim 1, wherein the model includes combined parameters of the tool and the flexible elongate device.

15. The medical system of claim 14, wherein the model further includes parameters of the actuator.

16. (canceled)

17. The medical system of claim 1, further comprising a sensor coupled to the flexible elongate device, and wherein the model is updated based on data from the sensor as the flexible elongate device traverses anatomy.

18. (canceled)

19. The medical system of claim 1, wherein the articulable body portion of the flexible elongate device includes a distal tip of the flexible elongate device; the expected position is a position for performing a medical procedure using the tool; the actual position is caused by movement of the tool within the lumen; and the control system is configured to operate the actuator to control articulation of the articulable body portion of the flexible elongate device to return the distal tip of the flexible elongate device to the expected position.

20. (canceled)

21. A method for controlling a flexible elongate device, the method comprising:
determining an expected position for an articulable body portion of a flexible elongate device, the flexible elongate device having a lumen extending therethrough for receiving one or more tools;
determining tool data associated with a tool insertable within the lumen of the flexible elongate device;
determining adjustment data based on a model of the flexible elongate device with the tool data to cause an actual position of the articulable body portion of the flexible elongate device to correspond with the expected position when the tool is inserted within the lumen; and
operating an actuator to control articulation of the articulable body portion of the flexible elongate device according to the adjustment data.

22. The method of claim 21, wherein the model defines a relationship between movement of the actuator and articulation of the articulable body portion of the flexible elongate device caused by the movement of the actuator.

23. The method of claim 21, further comprising generating encoder data regarding operational states of the actuator with an actuator encoder, wherein the model defines a relationship between the encoder data and articulation of the articulable body portion of the flexible elongate device caused by the operation of the actuator.

24.-25. (canceled)

26. The method of claim 21, further comprising generating torque data associated with operation of the actuator, wherein the model defines a relationship between the torque data of the actuator and articulation of the articulable body portion of the flexible elongate device caused by the operation of the actuator.

27.-28. (canceled)

29. The method of claim 21, wherein the adjustment data comprises at least one of:
a position command value to account for the deviated position of the articulable body portion of the flexible elongate device;
a feedforward control value determined using the model to account for the deviated position; or
a control value from a feedback controller based on an error of the expected position and the deviated position using the model.

30.-40. (canceled)

41. A non-transitory computer readable medium having instructions stored thereon that, when executed by a computing device, cause the computing device to:
determine an expected position for an articulable body portion of a flexible elongate device, the flexible elongate device having a lumen extending therethrough for receiving one or more tools;
determine tool data associated with a tool insertable within the lumen of the flexible elongate device;
determine adjustment data based on a model of the flexible elongate device with the tool data to cause an actual position of the articulable body portion of the flexible elongate device to correspond with the expected position when the tool is inserted within the lumen; and
operate an actuator to control articulation of the articulable body portion of the flexible elongate device according to the adjustment data.

42. The non-transitory computer readable medium of claim 41, further comprising generating: encoder data regarding operational states of the actuator with an actuator encoder or torque data associated with operation of the actuator, wherein the model defines a relationship between the encoder data or torque data and articulation of the articulable body portion of the flexible elongate device caused by the operation of the actuator.

43. The non-transitory computer readable medium of claim 41, wherein the adjustment data comprises at least one of:
a position command value to account for the deviated position of the articulable body portion of the flexible elongate device;
a feedforward control value determined using the model to account for the deviated position; or a control value from a feedback controller based on an error of the expected position and the deviated position using the model.
